(19) United States
(12) Patent Application Publication
(10) Pub. No.: US 2026/0170735 A1
(43) Pub. Date: Jun. 18, 2026

(54) REGULARIZED SPECT IMAGE RECONSTRUCTION WITH PLUG AND PLAY DENOISING PRIOR

(71) Applicant: Siemens Medical Solutions USA, Inc., Malvern, PA (US)

(72) Inventor: Xinhong Ding, Buffalo Grove, IL (US)

(21) Appl. No.: 19/333,557

(22) Filed: Sep. 19, 2025

Related U.S. Application Data
(60) Provisional application No. 63/733,525, filed on Dec. 13, 2024.

(51) Int. Cl. G06T 11/00; A61B 6/00; A61B 6/03; G06T 5/60; G06T 5/70; G06T 7/00

(52) U.S. Cl. CPC G06T 12/20; A61B 6/037; A61B 6/5258; G06T 5/60; G06T 5/70; G06T 7/0012; G06T 12/30; G06T 2207/10108; G06T 2207/20081; G06T 2207/20084; G06T 2207/30004; G06T 2207/30196; G06T 2210/41; G06T 2211/424

(57) ABSTRACT

For SPECT imaging, the reconstruction optimizes separately for the image or object and the regularizer, such as using alternating direction method of multipliers (ADMM). This framework provides for SPECT focused plug and play (PnP). Various denoisers may be used. The result is reconstruction specific to SPECT that has the hybrid (e.g., physics model with denoiser) benefits while minimizing complication.

### CROSS REFERENCE TO RELATED APPLICATIONS

This application claims the benefit of the filing date under 35 U.S.C. § 119 (e) of U.S. Provisional Patent Application Ser. No. 63/733,525, filed Dec. 13, 2024, which is hereby incorporated by reference in its entirety and relied upon.

### BACKGROUND

The present embodiments relate to reconstruction in Single Photon Computed Tomography (SPECT). Generally, there are three approaches to medical image reconstruction: (a) purely model based (b), purely data driven, and (c) hybrid of model based and data driven. Reconstruction approach (a) is the classical iterative or non-iterative methods currently used in most SPECT imaging systems. The reconstruction engine reconstructs by modelling of imaging physics. Reconstruction approach (b) uses supervised machine learning to learn the mapping from the measured data to the unknow object. This learning-based approach requires a large training data set, which is often difficult to obtain. The black-box nature of this approach makes it difficult to incorporate proper physics into the model.

Reconstruction approach (c) keeps the model-based reconstruction part and adds an advanced prior part through a plug and play (PnP) methodology. Many inverse problems in medical imaging, such as reconstruction, are il-posed and require proper regularizations. By adding any regularization term in an inverse problem, the optimization problem is more complicated. It is not straight forward to determine which prior should be used for specific applications. Thus, many inverse algorithms simply ignore the explicit regularization term, which removes the theoretical requirement to solve an il-posed inverse problem. For the PnP methodology, the prior is equivalent to an image denoiser (e.g., removing Gauss white noise from an image) in many cases. The prior term can also be developed separately and simply work together with the main inverse objective function though the PnP scheme.

### SUMMARY

By way of introduction, the preferred embodiments described below include methods, systems, instructions, computer product, and non-transitory computer readable storage media for SPECT imaging. The reconstruction optimizes separately for the image or object and the regularizer for SPECT. This framework provides for SPECT focused PnP. Various denoisers may be used. The result is reconstruction specific to SPECT that has the hybrid (e.g., approach (c)) benefits while minimizing complication.

In a first aspect, a method is provided for SPECT. Radiopharmaceutical emissions from a patient are detected with a SPECT detector. An image processor reconstructs the radiopharmaceutical emissions into an object. The reconstructing is formulated as first and second functions, the first function optimizing with respect to the object and the second function optimizing as a denoiser. The reconstructing alternates optimization with respect to the object and with respect to the denoiser. An image of the object as reconstructed is displayed.

In a second aspect, a method is provided for SPECT imaging. A SPECT detector detects emissions from a patient. An image processor reconstructs the emissions into an image. The reconstructing uses a physics model separate from a denoiser in a plug and play arrangement. The image is displayed.

In a third aspect, a SPECT system includes: a SPECT detector; a memory configured to store detected emissions from the SPECT detector; an image processor configured to formulate a regularized SPECT reconstruction as an optimization of two composite functions with a plug and play framework to derive a proximal alternating direction method of multipliers (ADMM) update in the optimization; and a display configured to display an image from the regularized SPECT reconstruction.

Other aspects are summarized below in the Illustrative Embodiments. Any of the various aspects may be used in any combination. Aspects for one set and/or type of set (e.g., method or system) may be used in other sets or types of sets (e.g., system, method, or non-transitory computer readable medium having stored thereon instructions (program product)).

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for SPECT imaging;

FIG. 2 illustrates an example arrangement of models or composite functions for SPECT reconstruction; and

FIG. 3 is a block diagram of one embodiment of a medical imaging system for SPECT reconstruction.

### DETAILED DESCRIPTION OF THE DRAWINGS AND PRESENTLY PREFERRED EMBODIMENTS

A regularized approach is used for iterative SPECT image reconstruction. This approach is aligned with the recent successes of the plug-and-play (PnP) methodology for general inverse problems but adopted specifically to the SPECT inverse problem. More precisely, the proposed approach contains a physics-based model reconstruction part, and an additional regularization part using one of various denoiser options as a prior. These two parts can be developed separately and work together through the PnP scheme. Unlike the traditional reconstructions algorithms in current SPECT that are based on classical Newtonian optimization theory for smooth functions, the proposed reconstruction algorithm utilizes the proximal operator and alternating direction method of multiplier (ADMM) from convex optimization for non-smooth functions.

The Bayesian SPECT reconstruction is formulated as a minimization problem of two composite functions. The PnP schema for this minimization problem of two composite functions is provided though the approach used in convex optimization. The proximal ADMM update algorithm is used for the regularized SPECT reconstruction. Due to this formulation, any of three types of PnP denoisers may be used to model the priors for SPECT regularized reconstruction.

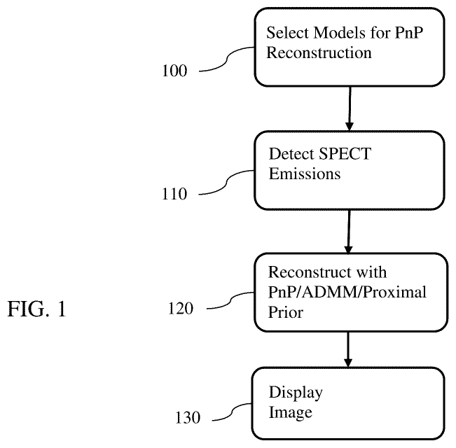

FIG. 1 shows one embodiment of a method for SPECT imaging. The PnP approach is used where separate or independent functions for the regularizer and physics model are used in optimization for reconstruction. This allows use of various physics models and/or regularizers and/or a less complicated solution.

The method of FIG. 1 is implemented using the system of FIG. 3, an image processor, a computer, a SPECT imager, a server, and/or another device. For example, a user using an input device and/or a controller using programming performs act 100, and SPECT system or imager performs acts 110-130. A computer (e.g., server, workstation, or processor), such as a computer of the SPECT imager, performs act 120. A SPECT detector (e.g., gamma camera) performs act 110, and a display screen is used for act 130.

The method is performed in the order shown (numerical or top-to-bottom/left-to-right), but other orders may be used. For example, act 100 may occur after act 110.

Additional, different, or fewer acts may be provided. For example, the display of the image in act 130 is not performed, instead saving the image or object in memory and/or transmitting the image or object over a computer network. As another example, acts for configuring the SPECT imager for the detection of act 110 are provided.

In act 100, the user or controller selects the regularizer (e.g., denoiser for SPECT) and/or physics model (i.e., forward model). Due to use of the PnP approach, either or both of the regularizer and physics model to be used in reconstruction may be selected. Since the PnP uses the physics model and regularization separate or independent of each other, one or both may be selected separately or independently.

The selection may be based on user preference, SPECT system configuration, SPECT system being used, imaging application (e.g., anatomy of interest and/or radiopharmaceutical (e.g., isotope) being used), and/or another variable. For example, the user selects a physics model and/or regularizer from a list or menu options. As another example, a controller selects the physics model based on an identification of the SPECT imager, the configuration for SPECT imaging, and/or application. Similarly, the regularizer is selected by the user and/or controller for various reasons, such as the isotope being used and/or the SPECT imaging application. Different regularizers may perform better for different isotopes and/or applications.

Different SPECT imagers may have different forward models modeling the physics of operation of the SPECT system. A given SPECT system may have different physics models based on configuration, such as the collimator used, dwell time, and/or sensitivity. The user or the controller selects the physics model to use for a given reconstruction.

Different regularizers may better represent distribution of emissions in different applications, for different isotopes, or for other reasons. A user may prefer one regularizer over another for a given situation. The user or the controller selects the regularizer to use for given reconstruction.

For SPECT, the regularizer can be formulated as a denoiser (e.g., removing Gauss white noise). The denoiser acts as a prior. The denoiser may be modeled in different ways. The selection may be between types of denoisers and/or between implementations of the same type of denoiser. Types of denoisers include an analytical denoiser, a denoiser trained through machine learning, and a denoising diffusion model. The denoiser or denoisers used and/or available for selection may be formulated as an analytical function, machine-learned function, and/or denoising diffusion model.

The analytical denoisers are non-learning based denoising methodology. One of the well-known examples is the Block-Matching 3D (BM3D) method that operates in two steps: the first step is to generate a basic estimate of the noise image using hard threshold; the second step is to perform actual denoising using a Wiener filter. A more general and powerful approach in the analytic category is the regularization by denoising (RED) in which a denoising engine is defined by a function φ:Rⁿ→Rⁿ that satisfies proper conditions. Using this denoising engine, the regularization term ρ(x) for the unknown image x can be explicitly expressed as:

$$ρ ( x ) = \frac{1}{2} {x}^{T} [ x - φ ( x ) ] \qquad ( 1 )$$

The denoisers trained through machine learning may use deep neural networks or another machine learning model. One example is the DnCNN that uses a convolution neural network (CNN) with the integration of residual learning and batch normalization. Other machine learning approaches and resulting data-based learning approaches may be used.

The denoising diffusion model (DDM) has forward and backward steps. In the forward step (learning step), DDM transforms the image data to a simple noise (e.g., Gauss noise). During the backward step (generative step), DDM reverses the process of the forward step, i.e., starting from a simple noise and generate the image data. Due to modeling the image data distribution, DDM does well for regularization in SPECT PnP image reconstruction.

The selected physics model and/or regularizer are plugged into the reconstruction. The physics model selected from the set of available physics models and the regularizer selected from the set of available regularizers provide selected functions for optimization in the reconstruction. The physics model and/or regularizer being used may be replaced based on selection. The replacement is independent for the physics model and the regularizer in the PnP formulation. These functions are used in the reconstruction to form the object in image or object space. Different ones of the physics models and/or regularizers result in different objects or optimized emission distributions from reconstruction. The image formed from the reconstruction may be different due to the selection.

In act 110, a SPECT detector detects emissions from the patient. Radiopharmaceutical emissions from the patient are detected. SPECT data are obtained. SPECT scanning is performed on a patient. The SPECT data are measurements of single photon emissions from a patient.

The SPECT data is obtained from scanning, from data transfer, or from memory. A SPECT system provides the SPECT data directly by scanning or indirectly by transfer or loading.

The activity concentration in a patient having received a radiotracer or radiotracers may be determined as part of reconstruction by a SPECT system. After ingesting or injecting the radiotracer or tracers into the patient, the patient is positioned relative to a SPECT detector, and/or the SPECT detector is positioned relative to the patient. Emissions from the radiotracer or tracers within the patient are detected over time as counts.

The SPECT detector may be rotated or moved relative to the patient, allowing detection of emissions from different angles and/or detector locations relative to the patient. Any dwell time at each view may be used, such as 5-10 seconds per view. The detector is then rotated or moved to another view, at which the detector dwells to detect emissions. Any number of views (typically 32, 64, 128) may be used. The emissions are detected over time at each view, and the different views correspond to different times or periods.

In act 120, the image processor reconstructs an object (representation of the distribution of radiopharmaceutical in the patient) from the radiopharmaceutical emissions. The detected emissions are reconstructed into object or image space representing activity concentration or distribution from or in the patient. An image or object is reconstructed from the emissions. The object corresponds to a distribution of emissions within the patient, which may or may not correspond to specific anatomy.

The reconstruction may use a system matrix or projection operators (e.g., physics or forward model) to describe the properties of the SPECT imaging system to iteratively improve a data model of an image object representing the SPECT data. The image object, which is defined in an object space, is a reconstruction of the SPECT data measured in a data space. The object space is the space in which the result of the image reconstruction is defined, and which corresponds, for example, to the 3D volume (i.e., field-of-view or “FOV”) that is scanned.

The reconstruction uses an optimization, such as a minimization or maximization. The optimization occurs over a number of iterations to fit the data model to the measured emissions. For each iteration, the image object is forward projected to data space. The resulting projection is compared to the acquired data. An objective function, such as a function for statistical or other distances (e.g., chi-square) is used to create some coefficients as part of the optimization, such as a fitting or solving using the conjugate gradient. The coefficients may be back projected into image space. The image object is altered accordingly, and then the process loops again for another iteration. The iterations continue until a stop criterion or criteria are met. Once complete (i.e., last iteration), the object from the last iteration is the reconstructed object or representation.

The object is reconstructed using all or most of the emissions detected from the different views. The object is tomographically reconstructed as a 3D object where the three dimensions are spatial. Dynamic objects (e.g., 4D) or a sequence of 3D objects may be reconstructed.

For PnP, the reconstruction is formulated and implemented using two different functions. One function optimizes with respect to the object (e.g., determining the object x from the measurements y), and the other function optimizes as the regularizer (e.g., denoiser). For example, one function represents or is the physics model of the SPECT detector and/or SPECT system, and the other function represents the regularization or prior (e.g., prior distribution of emissions). Since the physics model is separate from the regularizer (denoiser), both the physics model and the regularizer can be used as plug in functions in the PnP arrangement.

For SPECT, the regularizer can be formulated as a denoiser. The denoiser models a prior. In one implementation, the function for the denoiser includes or uses a proximal operator. Consider the following denoising problem: i.e., to recover the image x from noise measurement of:

$$y = x + e \qquad ( 2 )$$

where x, y, e∈ [symbol] ⁿ and e is a Gaussian noise with distribution N(0,σ²|). The maximum a posterior (MAP) estimator of x is given by:

$$\begin{matrix}{\hat{x}}_{MAP} = {argmin}_{x} \{ - log p ( y \vert x ) - log p ( x ) \} \\ = {argmin}_{x} \{ \frac{1}{2 {σ}^{2}} {‖ y - x ‖}^{2} + s ( x ) \}\end{matrix} \qquad ( 3 )$$

where ∥ \dot{∥} is the usual Euclidean norm of [symbol] ⁿ, p(y|x) is the likelihood function of y given x, and p(x) is the prior distribution of x. s(x)=−logp(x). Note that in deriving (3), the constant term that is independent of x is ignored.

For a proper closed convex function ƒ:Rⁿ→R∪ {+∞}, the proximal operator prox_{ƒ}: Rⁿ→Rⁿ of ƒ is defined by:

$$p r o {x}_{f} ( v ) := {arg min}_{x} \{ f ( x ) + \frac{1}{2} {‖ x - v ‖}^{2} \} \qquad ( 4 )$$

The scaled proximal operator for function λƒ is given as:

$$p r o {x}_{λ f} ( v ) := {arg min}_{x} \{ f ( x ) + \frac{1}{( 2 λ )} {‖ x - v ‖}^{2} \} \qquad ( 5 )$$

For scaling, λ>0.

By comparing equation (3) and equation (5), the denoising solution \hat{x}_{MAP} is written as:

$${\hat{x}}_{M A P} = p r o {x}_{{σ}^{2} s} ( y ) . \qquad ( 6 )$$

This provides a denoiser as the regularizer in SPECT where the denoiser uses the proximal operator.

For SPECT, the reconstruction is a regularized SPECT inverse problem. The optimization in the reconstruction is iterative. For each iteration, the optimization is alternated or sequentially performed with respect to the object and the denoiser. The reconstruction iteratively solves for the object using the physics model and then solves for the regularizer. The optimization of the physics (e.g., forward) model with respect to the object is carried out without the denoiser, and optimization of the denoiser is carried out without the object. The physics model as the corresponding function and the denoiser as the corresponding function are solvable independently, so either may be independently replaced as plug and play options.

In one implementation, the physics model and denoiser are used in the reconstruction in an alternating direction method of multipliers (ADMM) arrangement. Other arrangements for separately solving for the physics model and the denoiser in each iteration may be used. In ADMM, one function (physics model) is minimized, and then another function (denoiser) is solved for each iteration in the reconstruction. The physics model and the denoiser are sequentially and independently solved for each iteration of the reconstruction optimization.

A third function or model relates the object to the denoiser. In the PnP, the third function is solved after solving the functions for the physics model and the denoiser. Each iteration in the reconstruction solves for the relationship between the physics model and the denoiser. After determining the object and the prior, the relationship between them is determined.

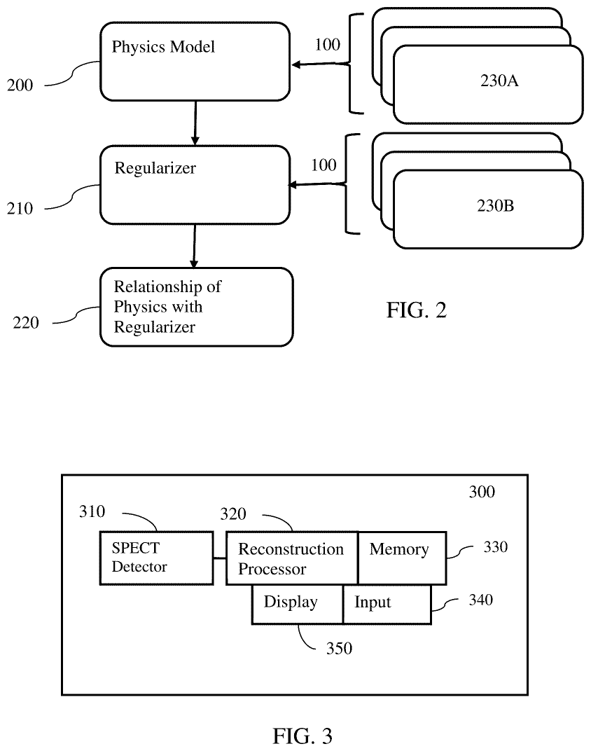

FIG. 2 shows an example. The physics model 200 is implemented as one function, the regularizer 210 is implemented as another function, and the relationship 220 is implemented as yet another function. Any of the functions may include more than one statement or equation. For PnP, the physics model 200 and the regularizer 210 are selected 100 from sets 230A and 230B of available functions.

In one implementation for regularized SPECT reconstruction as an inverse problem, ξ is a Poisson random variable with parameter λ, denoted by:

$$ξ ∼ Poisson ( λ ) , if \qquad ( 7 )$$

$$P ( ξ = k ) = \frac{{λ}^{k} {e}^{- λ}}{k !} , \qquad k = 0 , 1 , 2 , …$$

Let x={xₛ}, s=1, 2, . . . , n represent the SPECT object to be reconstructed, and y={yₜ}, t=1, 2, . . . , m represent the measured noise projection data. Then, according to the SPECT imaging physics, the measured projection data y can be approximated by a Poisson random variable:

$${y}_{t} ∼ Poisson ( {\sum }_{s} {a}_{ts} {x}_{s} ) \qquad ( 8 )$$

where (aₜₛ) represents the SPECT image formation weights from the object space to measured projection space.

The aim of SPECT reconstruction is to estimate x based on y. If {yₜ} for different t are independent, then according to Bayes' theorem, the following equalities result:

$$\begin{matrix}log P ( x \vert y ) = log P ( y \vert x ) + log P ( x ) + const \\ = {\sum }_{t} \{ {y}_{t} log ( {\sum }_{s} {a}_{ts} {x}_{s} ) - {\sum }_{s} {a}_{ts} {x}_{s} \} + log P ( x ) + const\end{matrix} \qquad ( 9 )$$

where the const includes the term that does not involve the variable x. Then, the maximize the posterior probability P(x|y) is equivalent to the following minimization problem (again, ignoring the constant term):

$${\hat{x}}_{M A P} = {arg min}_{x} \{ - log P ( x \vert y ) \} = {arg min}_{x} \{ f ( x ; y ) + g ( x ) \} \qquad ( 10 )$$

$$where :$$

$$f ( x ; y ) = {\sum }_{t} \{ {\sum }_{s} {a}_{ts} {x}_{s} - {y}_{t} log ( {\sum }_{s} {a}_{ts} {x}_{s} ) \} \qquad ( 11 )$$

$$and :$$

$$g ( x ) = - log P ( x ) \qquad ( 12 )$$

This is the minimization problem of two composite functions in convex optimization. To use the proximal ADMM algorithm, the unconstrained problem of equation (10) is transformed into a less complicated, constrained optimization problem. This can be done by introducing a new variable v and rewriting the problem of equation (10) as:

$${minimize}_{x , v} \{ f ( x ; y ) + g ( v ) \} \qquad ( 13 )$$

subject to v=x

In convex optimization, the augmented Lagrangian associated with the constrained problem of equation (13) is defined to be:

$${L}_{u , λ} ( x , v ; y ) := f ( x ; y ) + g ( v ) + < u , x - v > + \frac{λ}{2} {‖ x - v ‖}^{2} \qquad ( 14 )$$

where u is the Lagrangian multiplier vector, λ is an augmented Lagrangian multiplier scalar, and < > denotes the inner product of [symbol] ⁿ. The primal dual update method for the augmented Lagrangian is given by:
Initialization: u⁰∈Rⁿ, λ>0.
Iterative step: for any k=0,1,2, . . .

$$( {x}^{k + 1} , {v}^{k + 1} ) ∈ {arg min}_{x ∈ {R}^{n} , v ∈ {R}^{n}} \{ f ( x ; y ) + g ( v ) + \frac{λ}{2} {‖ x - v + \frac{1}{λ} {u}^{k} ‖}^{2} \} \qquad ( 15 )$$

$${u}^{k + 1} = {u}^{k} + λ ( {x}^{k + 1} - {v}^{k + 1} )$$

The augmented Lagrangian method as shown in equation (15) is in general not a method to be implemented. One of the main reasons is the coupling between the x variable and the v variable. The ADMM method handles this difficulty by replacing the exact minimization in the primal update step of equation (15) by one iteration of the alternating minimization method; that is, the objective function of equation (15) is first minimized w.r.t. x and then w.r.t v. This leads to the following update steps:
ADMM Updates for minimization problem (13)
Initialization: x⁰, v⁰∈Rⁿ, u⁰∈Rⁿ, λ>0.
Iterative step: for any k=0,1,2, . . .

$$\begin{matrix}( a ) {x}^{k + 1} ∈ {arg min}_{x} \{ f ( x ; y ) + \frac{λ}{2} {‖ x - {v}^{k} + \frac{1}{λ} {u}^{k} ‖}^{2} \} \\ ( b ) {v}^{k + 1} ∈ {arg min}_{v} \{ g ( v ) + \frac{λ}{2} {‖ {x}^{k + 1} - v + \frac{1}{λ} {u}^{k} ‖}^{2} \} \\ ( c ) {u}^{k + 1} = {u}^{k} + λ ( {x}^{k + 1} - {v}^{k + 1} )\end{matrix} \qquad ( 16 )$$

Note that in the ADMM update steps of equation (16), at each iteration k, the optimization of function (a) only involves x, and the optimization of function (b) only involves v. The optimization with respect to x and the optimization with respect to v can be carried out separately and work together through the PnP relationship step (c). The (b) part in equation (16) corresponds to a denoiser (see equations (3) to (6)).

By denoting

$$ρ = \frac{1}{λ} and {w}^{k} = \frac{1}{λ} {u}^{k} ,$$

then the update steps of equation (16) are simplified as:
ADMM update for minimization problem (13)
Initialization: x⁰, v⁰∈Rⁿ, w⁰∈Rⁿ, ρ>0.
Iterative step: for any k=0,1,2, . . .

$$\begin{matrix}( a ) {x}^{k + 1} ∈ {arg min}_{x} \{ f ( x ; y ) + \frac{1}{2 ρ} {‖ x - {v}^{k} + {w}^{k} ‖}^{2} \} \\ ( b ) {v}^{k + 1} ∈ {arg min}_{v} \{ g ( v ) + \frac{1}{2 ρ} {‖ {x}^{k + 1} - v + {w}^{k} ‖}^{2} \} \\ ( c ) {w}^{k + 1} = {w}^{k} + {x}^{k + 1} - {v}^{k + 1}\end{matrix} \qquad ( 17 )$$

or in the proximal operator notation:
Proximal ADMM updates for minimization problem (13)
Initialization: x⁰, v⁰∈Rⁿ, w⁰∈Rⁿ, ρ>0.
Iterative step: for any k=0,1,2, . . .

$$\begin{matrix}( a ) {x}^{k + 1} = p r o {x}_{ρ f} ( {v}^{k} - {w}^{k} ) \\ ( b ) {v}^{k + 1} = p r o {x}_{ρ g} ( {x}^{k + 1} + {w}^{k + 1} ) \\ ( c ) {w}^{k + 1} = {w}^{k} + {x}^{k + 1} - {v}^{k + 1}\end{matrix} \qquad ( 18 )$$

Replacing the SPECT imaging model ƒ(x, y) in equation (17) by the equation (11), then the ADMM updates for regularized SPECT reconstruction are as follows:
PROXIMAL ADMM updates for SPECT PnP
Inputs: Measured projection data y, Parameter ρ>0
Initialization: x⁰, v⁰, w⁰∈Rⁿ.
Iterative step: for any k=0,1,2, . . .

$$\begin{matrix}( a ) {x}^{k + 1} ∈ {arg min}_{x} \{ {\sum }_{t} \{ {\sum }_{s} {a}_{ts} {x}_{s} - {y}_{t} log ( {\sum }_{s} {a}_{ts} {x}_{s} ) \} + \frac{1}{2 ρ} {‖ x - {v}^{k} + {w}^{k} ‖}^{2} \} \\ ( b ) {v}^{k + 1} = p r o {x}_{ρ g} ( {x}^{k + 1} + {w}^{k} ) \\ ( c ) {w}^{k + 1} = {w}^{k} + {x}^{k + 1} - {v}^{k + 1}\end{matrix} \qquad ( 19 )$$

Equation 19 shows the function (a) as the physics model 200, the function (b) as the regularizer 210 with a proximal operator, and the function (c) as the relationship. Equation 19 provides a proximal ADMM regularized SPECT reconstruction formulation or configuration. The function (a) in equation (19) depends only on the SPECT measured data and the forward model (physics model), while function (b) is related to the image prior and can be replaced by a denoiser that is independent of the forward model. Thus, these two components functions (a) and (b) can be developed independent of each other and work together through the PnP schema as described by equation (19).

In act 130 of FIG. 1, the image or reconstruction processor generates an image from the output object. After optimization, the reconstruction outputs the object, such as 3D distribution of emissions from within the patient. The output of the reconstruction is used for imaging. The reconstruction outputs a volume representing the patient. An image of the reconstruction of the object is displayed.

The image or object is rendered or otherwise used to generate an image. For example, a multi-planar reconstruction and/or single slice image of a plane is generated. As another example, a 3D rendering, such as volume, surface, or another, of the volume is performed, and the resulting image is displayed.

The displayed image represents the functional information from the reconstruction. The emissions from the radiopharmaceutical represent function or uptake. An anatomical image is displayed with the functional image. For example, the functional image is overlaid on a CT image. The overlay may be colored for display on a gray scale CT image. Other combinations may be used. Alternatively, the SPECT image is displayed without an anatomical image.

FIG. 3 shows one embodiment of a SPECT system 300. The SPECT system 300 uses a PnP arrangement for reconstruction. Proximal ADMM is used, allowing PnP operation of the physics model and the denoiser. The arrangement of FIG. 2 is used. The method of FIG. 1 or another method is implemented.

The SPECT system 300 is a SPECT imager or scanner and includes a SPECT detector 310, reconstruction processor 320, a memory 330, user input 340, and a display 350. Additional, different, or fewer components may be provided. For example, a CT scanner is provided for zonal reconstruction and/or to provide anatomical imaging.

In one implementation, the reconstruction processor 320, memory 330, and/or display 350 are part of the SPECT imaging system. In alternative implementations, the reconstruction processor 320, memory 330, and/or display 350 are provided as a workstation, server, or computer separate from the SPECT detector 310. The memory 330 is part of a computer or workstation with the reconstruction processor 320 or is a remote database, such as a picture archiving and communications system (PACS).

The SPECT detector 310 includes one or more (e.g., two or dual head) detectors for detecting emitted radiation from within the patient. For SPECT, a gamma camera is used to detect. The gamma camera may be a cadmium zinc telluride (CZT) or another semi-conductor-based detector. A scintillation detector may be used. A collimator may be provided on or near the detector 310. The detector detects photon emissions. The photons are emitted from a tracer or radiopharmaceutical. The detector 310 detects the photons.

The SPECT detector 310 is mounted to or connected with a gantry. A motor moves the gantry to position the detector or detectors at different positions relative to the patient. The detector(s) are rotated or moved through a sequence of views (positions) relative to the patient. At each position, the detector(s) are held in place during a dwell time, detecting emissions. The detector(s) detect events from the same or different locations of the patient during the dwell time. By using the detected emissions from multiple views, a sufficient emission for reconstruction may be provided.

The user input 340 is a keyboard, mouse, trackball, touch screen, touch pad, knob, dial, slider, button, capacitive sensor, and/or another user input device for the user to communicate with the reconstruction processor 320. The operator inputs selection of the application, SPECT imaging configuration, radiopharmaceutical to be used, denoiser selection, physics model selection, and/or other information. The information is used by the reconstruction processor 320 to plug in (e.g., replace) the physics model and/or denoiser. Alternatively, the reconstruction processor accesses the information through the memory 330 or another source without using the user input 340.

The memory 330 is a random-access memory, graphics processing memory, video random access memory, system memory, cache memory, hard drive, optical media, magnetic media, flash drive, buffer, database, combinations thereof, or other now known or later developed memory device for storing data. The memory 330 stores detected emissions (e.g., SPECT detected event data from multiple positions of the detector 310 relative to the patient). Other information may be stored, such as user selections, SPECT configuration, radiopharmaceutical identification, application, anatomical information (e.g., CT volume), zone information (e.g., zone map), segmentation information, projections, fitting information, and/or reconstruction information. The memory 330 stores data as processed, such as storing an updated image object, function solutions, projection operators or system matrix, and/or other information.

The memory 330 or other memory is a non-transitory computer readable storage medium storing data representing instructions executable by the programmed reconstruction processor 320 for SPECT reconstruction. The instructions for implementing the processes, methods and/or techniques discussed herein are provided on computer-readable storage media or memories, such as a cache, buffer, RAM, removable media, hard drive, or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code, and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The reconstruction processor 320 is an image processor for SPECT reconstruction. The reconstruction processor 320 is a general processor, central processing unit, control processor, graphics processor, digital signal processor, application specific integrated circuit, field programmable gate array, artificial intelligence processor, digital circuit, analog circuit, timing circuit, combinations thereof, or other now known or later developed device for reconstructing a patient volume from detected emissions. The reconstruction processor 320 is a single device or multiple devices operating in serial, parallel, or separately. The reconstruction processor 320 is specifically designed or provided for reconstruction but may be a main or general processor of a computer, such as a laptop or desktop computer, or may be a processor for handling tasks in a larger system. The reconstruction processor 320 may perform other functions than SPECT reconstruction, such as rendering images.

The reconstruction processor 320 is configurable. The reconstruction processor 320 is configured by software, firmware, and/or hardware. Different software, firmware, and/or instructions are loaded or stored in memory 330 for configuring the reconstruction processor 320.

The reconstruction processor 320 is configured to formulate a regularized SPECT reconstruction as an optimization of two composite functions with a plug and play framework to derive a proximal alternating direction method of multipliers (ADMM) update in the optimization. For example, the functions of equation (19) are used for the reconstruction. One of the two composite functions is a denoiser as a regularizer of the regularized SPECT reconstruction. The denoiser is implemented with a proximal operator of the proximal ADMM.

The composite function for regularization may be selected from a set of such functions, such as a set that includes one or more analytical denoisers, one or more denoisers trained through machine learning, and/or one or more denoising diffusion models.

Another of the composite functions implements or includes a physical model for the SPECT detector 310 and corresponding SPECT system 300. The function and corresponding physical model (e.g., forward model) may be selected from a set of such models.

The selection of the denoiser and/or physical model is part of the PnP framework. The functions implementing the denoiser and the physical model operate independently of each other in each iteration of the reconstruction optimization, allowing for the PnP scheme. A relationship function links the two composite functions.

The reconstruction processor 320 or another image processor generates an image from the reconstructed object. Multiplanar reconstruction, slice (cross-section), or 3D rendering may be used to generate the SPECT image.

The display 350 is a monitor, LCD, plasma, touch screen, printer, or another device for displaying the SPECT image for viewing by a user. The display 350 shows one or more images representing function, such as uptake or activity concentration. The image(s) represent a distribution of the radionuclide in the patient based on detected emissions from the SPECT system 300. The image is from the regularized SPECT reconstruction.

The following is a list of non-limiting Illustrative Embodiments disclosed herein. The illustrative embodiments below summarize aspects or features of the first, second, and third aspects in the summary above. Aspects or features used for one type of illustrative embodiment (e.g., method or system) may be used in other types.

Illustrative Embodiment 1. A method for single photon emission computed tomography (SPECT) imaging, the method comprising: detecting radiopharmaceutical emissions from a patient, the radiopharmaceutical emissions detected with a SPECT detector; reconstructing, by an image processor, the radiopharmaceutical emissions into an object, the reconstructing formulated as first and second functions, the first function optimizing with respect to the object and the second function optimizing as a denoiser, the reconstructing alternating optimization with respect to the object and with respect to the denoiser; and displaying an image of the object as reconstructed.

Illustrative Embodiment 2. The method of Illustrative Embodiment 1, wherein the alternating optimization comprises an alternating direction method of multipliers (ADMM) where the first function is minimized and then the second function for every iteration in the reconstructing.

Illustrative Embodiment 3. The method of any of Illustrative Embodiments 1-2, wherein the first function represents a physics model of the SPECT detector, and the second function represents a regularizer such that the reconstructing iteratively solves for the object using the physics model and the regularizer.

Illustrative Embodiment 4. The method of Illustrative Embodiment 3, wherein the regularizer comprises a prior distribution.

Illustrative Embodiment 5. The method of any of Illustrative Embodiments 1-4, wherein the first function optimizing with respect to the object is carried out without the denoiser and the second function optimizing as the denoiser is carried out without the object.

Illustrative Embodiment 6. The method of Illustrative Embodiment 5, further comprising handling the first function and the second function as plug and play options such that the first function and/or the second function is replaceable independently.

Illustrative Embodiment 7. The method of Illustrative Embodiment 6, wherein the second function is selectable from a set of regularizers formulated as analytical functions, machine-learned functions, and/or denoising diffusion models.

Illustrative Embodiment 8. The method of any of Illustrative Embodiments 1-7, further comprising replacing the first function and/or the second function independently in a plug and play formulation.

Illustrative Embodiment 9. The method of Illustrative Embodiment 8, wherein the plug and play formulation comprises a third function relating the object to the denoiser, and wherein reconstructing comprises solving the third function after solving the first and second functions for each iteration in the reconstructing.

Illustrative Embodiment 10. The method of any of Illustrative Embodiments 1-9, wherein the second function comprises a proximal operator.

Illustrative Embodiment 11. A method for single photon emission computed tomography (SPECT) imaging, the method comprising: detecting emissions from a patient with a SPECT detector; reconstructing, by an image processor, the emissions into an image, the reconstructing using a physics model separate from a denoiser in a plug and play arrangement; and displaying the image.

Illustrative Embodiment 12. The method of Illustrative Embodiment 11, wherein the physics model and denoiser are used in the reconstructing in an alternating direction method of multipliers (ADMM) arrangement.

Illustrative Embodiment 13. The method of any of Illustrative Embodiments 11-12, wherein the reconstructing uses the denoiser as a prior in a proximal operator.

Illustrative Embodiment 14. The method of any of Illustrative Embodiments 11-13, further comprising selecting the denoiser from a set of denoisers and plugging the denoiser into the reconstructing with the physics model.

Illustrative Embodiment 15. The method of Illustrative Embodiment 14, wherein selection comprises selecting where the set includes an analytical denoiser, a denoiser trained through machine learning, and a denoising diffusion model.

Illustrative Embodiment 16. The method of any of Illustrative Embodiments 11-15, wherein the reconstructing comprises iterative optimization, the physics model and the denoiser sequentially and independently solved for each iteration of the iterative optimization, each of the iterations including solving a relationship between the physics model and the denoiser after solving the physics model and the denoiser.

Illustrative Embodiment 17. A single photon emission computed tomography (SPECT) system comprising: a SPECT detector; a memory configured to store detected emissions from the SPECT detector; an image processor configured to formulate a regularized SPECT reconstruction as an optimization of two composite functions with a plug and play framework to derive a proximal alternating direction method of multipliers (ADMM) update in the optimization; and a display configured to display an image from the regularized SPECT reconstruction.

Illustrative Embodiment 18. The SPECT system of Illustrative Embodiment 17, wherein one of the two composite functions comprises a denoiser as a regularizer of the regularized SPECT reconstruction, the denoiser comprising a proximal operator of the proximal ADMM.

Illustrative Embodiment 19. The SPECT system of any of Illustrative Embodiments 17-18, wherein a first one of the two composite functions comprises a physical model selected for the SPECT detector and a second one of the two composite functions comprises a denoiser selected from an analytical denoiser, a denoiser trained through machine learning, and a denoising diffusion model, the selections part of the plug and play framework where the first and second composite functions operate independently of each other in each iteration of the optimization.

Illustrative Embodiment 20. The SPEC system of Illustrative Embodiment 19, wherein the optimization further comprises a relationship function linking the two composite functions.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for single photon emission computed tomography (SPECT) imaging, the method comprising:
detecting radiopharmaceutical emissions from a patient, the radiopharmaceutical emissions detected with a SPECT detector;
reconstructing, by an image processor, the radiopharmaceutical emissions into an object, the reconstructing formulated as first and second functions, the first function optimizing with respect to the object and the second function optimizing as a denoiser, the reconstructing alternating optimization with respect to the object and with respect to the denoiser; and
displaying an image of the object as reconstructed.

2. The method of claim 1, wherein the alternating optimization comprises an alternating direction method of multipliers (ADMM) where the first function is minimized and then the second function for every iteration in the reconstructing.

3. The method of claim 1, wherein the first function represents a physics model of the SPECT detector, and the second function represents a regularizer such that the reconstructing iteratively solves for the object using the physics model and the regularizer.

4. The method of claim 3, wherein the regularizer comprises a prior distribution.

5. The method of claim 1, wherein the first function optimizing with respect to the object is carried out without the denoiser and the second function optimizing as the denoiser is carried out without the object.

6. The method of claim 5, further comprising handling the first function and the second function as plug and play options such that the first function and/or the second function is replaceable independently.

7. The method of claim 6, wherein the second function is selectable from a set of regularizers formulated as analytical functions, machine-learned functions, and/or denoising diffusion models.

8. The method of claim 1, further comprising replacing the first function and/or the second function independently in a plug and play formulation.

9. The method of claim 8, wherein the plug and play formulation comprises a third function relating the object to the denoiser, and wherein reconstructing comprises solving the third function after solving the first and second functions for each iteration in the reconstructing.

10. The method of claim 1, wherein the second function comprises a proximal operator.

11. A method for single photon emission computed tomography (SPECT) imaging, the method comprising:
detecting emissions from a patient with a SPECT detector;
reconstructing, by an image processor, the emissions into an image, the reconstructing using a physics model separate from a denoiser in a plug and play arrangement; and
displaying the image.

12. The method of claim 11, wherein the physics model and denoiser are used in the reconstructing in an alternating direction method of multipliers (ADMM) arrangement.

13. The method of claim 11, wherein the reconstructing uses the denoiser as a prior in a proximal operator.

14. The method of claim 11, further comprising selecting the denoiser from a set of denoisers and plugging the denoiser into the reconstructing with the physics model.

15. The method of claim 14, wherein selection comprises selecting where the set includes an analytical denoiser, a denoiser trained through machine learning, and a denoising diffusion model.

16. The method of claim 11, wherein the reconstructing comprises iterative optimization, the physics model and the denoiser sequentially and independently solved for each iteration of the iterative optimization, each of the iterations including solving a relationship between the physics model and the denoiser after solving the physics model and the denoiser.

17. A single photon emission computed tomography (SPECT) system comprising:
a SPECT detector;
a memory configured to store detected emissions from the SPECT detector;
an image processor configured to formulate a regularized SPECT reconstruction as an optimization of two composite functions with a plug and play framework to derive a proximal alternating direction method of multipliers (ADMM) update in the optimization; and
a display configured to display an image from the regularized SPECT reconstruction.

18. The SPECT system of claim 17, wherein one of the two composite functions comprises a denoiser as a regularizer of the regularized SPECT reconstruction, the denoiser comprising a proximal operator of the proximal ADMM.

19. The SPECT system of claim 17, wherein a first one of the two composite functions comprises a physical model selected for the SPECT detector and a second one of the two composite functions comprises a denoiser selected from an analytical denoiser, a denoiser trained through machine learning, and a denoising diffusion model, the selections part of the plug and play framework where the first and second composite functions operate independently of each other in each iteration of the optimization.

20. The SPEC system of claim 19, wherein the optimization further comprises a relationship function linking the two composite functions.
